(19) United States
(12) Patent Application Publication
(10) Pub. No.: US 2015/0209399 A1
(43) Pub. Date: Jul. 30, 2015

(54) Methods For Enhacement Of Dehydroepiandrosterone Using Green Coffee Bean Extract

(71) Applicant: Applied Food Sciences, Inc., Austin, TX (US)

(72) Inventor: Chrstine C. Fields, Janesville, WI (US)

(21) Appl. No.: 14/562,642

(22) Filed: Dec. 5, 2014

Related U.S. Application Data
(60) Provisional application No. 61/912,263, filed on Dec. 5, 2013.

(51) Int. Cl. A61K 36/74; A61K 31/216; A61K 31/192

(52) U.S. Cl. CPC A61K 36/74; A61K 31/192; A61K 31/216

(57) ABSTRACT

A method for elevating naturally recirculating dehydroepiandrosterone (DHEA) levels in a human subject, the method including generating a green coffee extract containing at least 20% chlorogenic acids by dry weight, generating a composition which includes the green coffee extract and is administrable to a human subject, and administering a therapeutically effective amount of the composition to the human subject, thereby elevating naturally recirculating DHEA levels.

### STATEMENT OF PRIORITY

The present application claims priority to U.S. Provisional Application No. 61/912,263, titled “Methods For Enhancement Of Dehydroepiandrosterone Using Green Coffee Bean Extract” and filed Dec. 5, 2013.

### FIELD OF THE INVENTION

The present disclosure relates to methods to enhance or increase naturally recirculating levels of dehydroepiandrosterone in human subjects using green coffee bean extract.

### BACKGROUND OF THE INVENTION

Cortisol and dehydroepiandrosterone (DHEA) are major steroids of the adrenal gland. Dehydroepiandrosterone, also known as prasterone, 3-hydroxyandrost-5-en-17-one, dehydroisoandrosterone, trans-dehydroandrosterone, or Δ.sup.5-androsten-3-βol-17-one (referred to hereinafter as “DHEA”), is a 17-ketosteroid, which is quantitatively one of the major adrenocortical steroid hormones present in the metabolism of humans and other mammals. S. Budavari, ed., Merck Index, Eleventh Edition (1989). This endogenous androgenic steroid has been shown to have a myriad of biological activities. An assortment of prior art has recognized the plethora of beneficial effects of DHEA, its sulfate ester (DHEA-S) and salts thereof. DHEA is readily interconvertible in vivo with DHEA-S through the action of intracellular sulfatases.

Cortisol is the major glucocorticoid in humans and has a wide range of influences on metabolism, immunoregulation, vascular responsiveness, cognition, and behavior. DeGroot L J, Jameson J L. Endocrinology. 5th ed. Philadelphia: Elsevier Saundrs; 2005. Pp. 2287; Johnson K L, Rn C R. The Hypothalamic-pituitary-adrenal axis in critical illness. AACN Clin Issues 2006; 17:39-49. [PubMed]. Cortisol also has impact on numerous pathological conditions including inflammatory autoimmune disorder, atopical conditions, metabolic syndrome, and depression. Jefferies W M. Cortisol and immunity. Med Hypotheses. 1991; 34:198-208. [PubMed]; Schleimer R P. Interactions between the hypothalamic-pituitary-adrenal axis and allergic inflammatory disease; Allergy Clin Immunol. 2000; 106(SSuppl); 270-274. [PubMed]; Rosmond R, Dallman M F, Bjornatorp P. Stress-related cortisol secretion in men; relationship with abdominal obesity and endocrine, metabolic and hemodynamic abnormalities. J Clin Endocrinol Med 1998; 83:1853-1859. [PubMed]; de Kloet E R. Hormones, brain and stress. Endocr Regul. 2003; 37:51-68. [PubMed].

Adrenal fatigue is prevalent in today's society, though it was first described in textbooks over a century ago. The term refers to exhaustion of adrenal glands, which secrete two hormones related to stress level: Cortisol and DHEA. Affected individuals have elevated Cortisol levels and depressed DHEA levels, and may suffer from a broad spectrum of non-specific yet debilitating symptoms, such as low energy, sleep problems, weight gain, memory loss, and susceptibility to infections. For example, commercially available extracts (e.g. Relora®) although capable of improving Cortisol and DHEA levels in the body are limited in use due to the side effects. Garrison et al., Alternative Therapies in Heath and Medicine 2006, 12(1):50-54. Hence, there is a need for compositions which improve the delivery and/or reduce the side effects of these medicinal extracts.

In U.S. Pat. No. 4,920,115 to Nestler et al., oral dosages of DHEA given to healthy male individuals were shown to reduce body fat mass, increase muscle mass, lower LDL cholesterol levels without affecting HDL cholesterol levels, and not affect tissue sensitivity to insulin in human patients. Nestler et al. described the use of pharmaceutical preparations of DHEA as a preventative means to avoid development of atherosclerosis.

U.S. Pat. Nos. 5,110,810 and 5,162,198 issued to Eich et al., disclose methods for treating human beings with pharmacological quantities of DHEA, resulting in increased serum DHEA and DHEA-S in their blood, which lowers rates of platelet aggregation. By reducing the rate of platelet aggregation, the incidence of morbidity and mortality from vascular events such as myocardial infarction and stroke, as well as the occurrence of restenosis following vascular interventions, can be significantly reduced.

U.S. Pat. No. 4,835,147 to Roberts demonstrated that administration of DHEA or its therapeutically acceptable salts to individuals ameliorated symptoms of prostatic hypertrophy, certain symptoms of menopause, particularly those related to nervous system dysfunction, and of psycho-sexual dysfunction, symptoms such as inhibited sexual desire, inhibited sexual excitement and inhibited orgasm.

Other widely varying medical uses for DHEA have been reported. U.S. Pat. No. 4,628,052, issued to Peat, reports using either an oral or topical preparation of DHEA to treat rheumatoid arthritis, osteo-arthritis and arthritis associated with psoriasis and with lupus and other auto-immune diseases, and also for treating non-specific joint pain associated with stress or incidental to other ailments.

DHEA compounds have also been established to have a beneficial effect as an anti-diabetic agent. See U.S. Pat. No. 4,518,595 to Coleman et al. In the medical literature, many favorable reports of medical benefits to individuals due to increased levels of DHEA and its sulfate ester, DHEA-S, have been reported as well. Geriatrics 37:157 (1982) stated that DHEA was a “miracle” drug, which may prevent obesity, aging, diabetes mellitus and heart disease. Barrett-Conner et al. produced studies which revealed an inverse relationship between cardiovascular death and serum DHEA-S levels in adult men. N. Engl. J. Med. 315:1519 (1986). Arad et al. in Arteriosclerosis 9:159 (1989) and Gordon et al. in J. Clin. Invest 82:712 (1988) both describe the reduction of atherosclerosis plaque formation by DHEA.

One of the most important uses of DHEA has been to improve the immune response in human beings. U.S. Pat. No. 5,077,284, issued to Loria et al., describes the administration of DHEA, either orally or by subcutaneous injection, to provide very high levels of protection against viral, bacterial, fungal or parasitic infections in immuno-compromised animals and humans. The experimental animal data, described by Loria et al., demonstrated that in infection (100,000 plaque forming units/animal) of a human coxsackievirus B4 strain, which causes mortality in about 90% of infected animals, mortality was reduced to 37% when animals were treated with DHEA. Moreover, Loria et al. demonstrated that administration of DHEA induced an 80% elevation in the number of antibody forming cells within the animal. In virus infected and DHEA treated animals, there was also an elevation in the number of monocyte cells, the particular white blood cells associated with a resistance to coxsackievirus infection. This elevation was not observed in uninfected animals that were treated with DHEA. This observation demonstrates that DHEA can be used to up-regulate the host immune response to virus infection, by increasing the number of antibody forming cells, elevating the number of white blood cells associated with resistance to virus infection and markedly reducing virus induced mortality.

Although DHEA is the most abundantly produced adrenal steroid and serum concentrations of its sulfate ester, DHEA sulfate (DHEA-S), are approximately 20 fold higher than those of any other circulating steroid hormone, levels of this hormone begin to decline within individuals during the second decade of life, reaching 5% of the original level in the elderly.

Peak serum DHEA and DHEA-S levels occur when a patient is approximately 25 years old and decline over the ensuing decades. Ohrentreich et al. found that mean DHEA-S levels and ranges for adult men were as follows: Ages 25-29 (3320 ng/ml); ages 45-49 (1910 ng/ml); ages 65-69 (830 ng/ml). See J. Clin. Endocrinol. Metab., 59:551 (1984). Similar age related decline in serum DHEA-S levels were found to occur in women. Correspondingly, the incidence of cardiovascular disease in human beings increases with age, thus suggesting an epidemiological relationship between serum DHEA and DHEA-S levels in cardiovascular disease. In Barrett-Conner et al., supra, the baseline DHEA-S levels of 242 middle aged men (ages ranging between 50 and 79 years) was compared to the subsequent 12 year mortality rate of the men from any cause, from cardiovascular disease, and from ischemic heart disease. DHEA-S levels were significantly lower in men with a history of heart disease compared to those without. In men with no history of heart disease, the age-adjusted relative risk associated with DHEA-S levels below 140 μg/dl was 1.5 (p NS) for death from any cause, 3.3 (p&It;0.05) for deaths from cardiovascular disease, and 3.2 (p&It;0.05) for deaths from ischemic heart disease. An increase in DHEA-S level of 100 μg/l had a 48% reduction in mortality (adjusted for other risk factors) from cardiovascular disease (p&It;0.05).

As more sophisticated hormone assay techniques for saliva were developed, more researchers became involved in the study of age and stress-related adrenal steroid circadian rhythm changes. Saliva provides a useful sample for cortisol/DHEA measurement in many cases because the level of steroids in saliva reflects that in blood. Recently, the analysis of salivary steroids is becoming a widely accepted screening tool for adrenal or gonadal function. Individual circadian rhythm has become more important than the absolute hormonal concentrations in disease diagnosis. Studies confirmed that salivary steroid levels reflect that of serum levels.

Further Eich et al. supra, demonstrated that treating human beings with pharmacological quantities of DHEA resulted in increased serum levels of DHEA and DHEA-S. Eich et al. performed in vivo experiments using a test group of 10 male human being subjects. In these experiments, DHEA was administered in a double-blind placebo controlled trial in an amount of 300 mg of DHEA per day in the form of 100 mg capsules taken orally 3 times a day. The study found that the initial baseline serum DHEA prior to conducting the experiment was 5.83+/−3.9 μg/ml, and the mean serum DHEA during the second week of investigation for the placebo group was 5.58+/−4.1 μg/ml. The mean serum DHEA for the treated group during the second week investigation was 28.7+/−13.9 μg/ml. In addition, the baseline serum DHEA-S was 316.2 μg/dl and during the second week, the mean serum DHEA-S level was 260.5+/−56.7 μg/dl in the placebo group, and 1451.9+/−56.7 μg/dl in the DHEA group. Elevation of serum DHEA-S levels when a patient is receiving only supplemental DHEA suggested that DHEA-S serves as a storage pool for DHEA, which is the active form of the hormone. The rate of platelet aggregation for the human subjects participating in this study was examined prior to treatment with supplemental DHEA and was again tested on three different occasions during the second week of an investigation. Four of the five test subjects who received the DHEA supplement demonstrated a slower rate of aggregation and a requirement for higher concentration of arachidonic acid to initiate. aggregation. Thus, the elevated serum DHEA level slowed platelet aggregation which can significantly reduce the incidence of morbidity and mortality from vascular events such as myocardial infarction and stroke.

An extract of green coffee beans (commercially available under the trade name GCA®) contains a profile of at least 20 w/w % chlorogenic acids that have been shown to have a host of health benefits from LDL oxidation reduction, enhanced endothelial function aiding in hypertension reduction and general antioxidant function for reduced oxygen species activity in vivo. In addition, these specific polyphenols have been shown to have suppressive effects on the glucose 6-phosphatase pathway. This pathway is our body's primary pathway for the regulation and uptake of glucose into the cell walls. It is speculated or deduced that by controlling the regulation of glucose in this way we can assist the body with weight management, fatty acid synthesis activity and positively impact insulin activity.

While historical research supported the role these chlorogenic acids have on lowering the plasma glucose levels in response to oral dose administration, the science and clinical data was not clear on the specific mechanism of action associated with the secondary benefits seen in human subjects such as weight loss, increased energy, more satisfied disposition and mental health, increased sexual drive, etc. upon administration of the extract. Furthermore, synthetic derivatives of the chlorogenic acids did not carry any such secondary benefits in response to lower glycemic response.

Citation of the above documents is not intended as an admission that any of the foregoing is pertinent prior art. All statements as to the date or representation as to the contents of these documents is based on the information available to the applicants and does not constitute any admission as to the correctness of the dates or contents of these documents. Further, all documents referred to throughout this application are incorporated in their entirety by reference herein.

### SUMMARY OF THE INVENTION

The present disclosure provides methods for enhancing or restoring DHEA in a subject human without the use of pharmaceutical preparations containing DHEA, its sulfate ester DHEA-S, or salts thereof.

Specifically, it has been discovered that by increasing naturally recirculating DHEA levels, GCA® administration can provide a source of enhanced adrenal and metabolic function, increase lean body mass by enhanced testosterone leading to increased muscle formation, increase libido, ovary function and elevated mood and brain receptivity.

One object of the present disclosure is a method for elevating naturally recirculating dehydroepiandrosterone (DHEA) levels in a human subject, the method including generating a green coffee extract containing at least 20% chlorogenic acids by dry weight, generating a composition which includes the green coffee extract and is administrable to a human subject, and administering a therapeutically effective amount of the composition to the human subject, thereby elevating naturally recirculating DHEA levels.

Another object of the present disclosure is to provide compositions for elevating naturally recirculating levels of DHEA, which compositions comprise effective amounts of CGA®, said effective amounts ranging from approximately 1 mg to approximately 4000 mg per dosage and administered to the human subjects at least twice a day for a period of at least two weeks. Another object is to provide methods for elevating naturally recirculating levels of DHEA in a human subject by using said compositions.

Further, the present disclosure provides a means to control, enhance or increase the natural recirculating DHEA hormone levels by the administration of GCA®, green coffee bean extract, which contains at least 20% total chlorogenic acids and related compounds having the following composition by weight percent:

3-caffeylquinic acids (3-CQA) ranging from approximately 2% to approximately 15%;
5-caffeylquinic acids (5-CQA) ranging from approximately 5% to approximately 25%;
4-caffeylquinic acids (4-CQA) ranging from approximately 2% to approximately 15%;
5-feruloylquinic acids (5-FQA) ranging from approximately 1% to approximately 12%;
3,4-dicaffeylquinic acids (3,4-diCQA) ranging from approximately 1% to approximately
12%;
3,5-dicaffeylquinic acids (3,5 diCQA) ranging from approximately 1% to approximately
12%; and
4,5-dicaffeylquinic acids (4,5 diCQA) ranging from approximately 1% to approximately 12%.

According to still further features in the described preferred embodiments, the composition described herein is in the form of a supplement, beverage, food, tea, a tincture, a concoction, an infusion tablet, a capsule, a pill, a bar, a chewable gum, a dissolvable oral strips, a lotion, a powder or granules.

According to still further features in the described preferred embodiments, there is provided a dietary and/or pharmaceutical composition comprising an herbal composition described herein and a dietetically and/or pharmaceutically acceptable excipient.

It has been found that the greatest increase in saliva DHEA levels occurred in patients treated with the dosage of GCA® 350 mg daily for a period of approximately two (2) weeks. Administered orally GCA has been shown to increase DHEA levels by as much as 300% in human subjects. Subsequently, this elevation in DHEA levels has been associated with increased energy and metabolism, weight loss, increased subject testosterone levels and lean muscle mass, feelings of happiness, satisfaction and sexual desire as well as elevated serotonin levels. Additionally, elevation of DHEA has been shown to elevate testosterone levels, and thus help treat negative symptoms associated with low testosterone levels, including loss of muscle mass. In other embodiments, cortisol levels may be reduced and conditions associated with mood disorders or neurotransmitter functions may be treated or alleviated.

According to one embodiment of the present disclosure, GCA® can also be applied topically as a serum, cream, lotion, oil, gel or patch. Applied topically, GCA® has been shown to enhance cell turnover and decrease the physical signs of skin aging, such as skin UV damage, wrinkles, lines, and scarring.

### BRIEF DESCRIPTION OF THE DRAWINGS

The following figures are included to illustrate certain aspects of the present invention, and should not be viewed as an exclusive embodiments. The subject matter disclosed is capable of considerable modification, alteration, and equivalents in form and function, as will occur to one having ordinary skill in the art and the benefit of this disclosure.

FIG. 1 is a chart of a Visia® analysis relative to control group for each skin parameter measured, according to one or more embodiments.

FIG. 2 depicts statistics for a control group and a coffee extract group, according to one or more embodiments.

FIG. 3 depicts product performance for various skin parameters, according to one or more embodiments.

### DETAILED DESCRIPTION

### Examples

The following examples are illustrative of the present disclosure and parts and percentages are by dry weight unless otherwise indicated. It should be noted that these examples are only that—examples—a wide range of conditions, which together with the above descriptions, illustrate the invention in a non limiting fashion.

Twelve human subjects, 6 female and 6 male subjects were recruited to evaluate the affect of oral administration of GCA® on recirculating hormones. The twelve subjects were initially screened for any preexisting conditions, potential drug interactions, health issues and current supplement and or drug usage. All subjects were in good health and currently not taking any sort of treatments that were known to have an impact on the tested hormones. Each subject was requested to provide saliva samples for an initial hormone's testing to determine baseline hormone levels. A complete hormone panel test which includes Estrogen, Testosterone, DHEA, and am, mid-pm and pm Cortisol levels were completed by Labrix Clinical Services. After baseline readings were completed GCA® having the following chlorogenic acid composition was orally administered at 350 mg, twice daily for four weeks:

### 3-CQA=6.78%

### 5-CQA=20.9%

### 4-CQA=8.31%

### 5-FQA=3.62%

3,4-diCQA=3.65%
3,5-diCQA=4.56%
4,5-diCQA=2.88%

### Example 1

After four weeks, a second set of saliva samples were submitted by each subject for additional hormone testing on the same parameters measured during the baseline readings. The baseline and subsequent results after the 4 weeks of GCA administration are shown in Tables 1 and 2.

Five of the six female subjects also completed a quality of life questionnaire to determine if they observed any changes in their physical and or emotional state during the trial period. The scores were ranked 0-10 with 0 being the lowest state and 10 being the highest. The female subjects were asked to rank their energy levels, as determined by their ability to maintain energy throughout the day, satisfaction in their overall weight, energy and mood, sex drive by their number and days they initiate sexual intimacy with their life partner, motivation by their overall willingness to take on new projects, try new things or aide others in performing a task and happiness by their overall feeling of being in a happy state of mind throughout the day. The quality of life scores for all five subjects is provided in Table 3.

HORMONE PANEL RESULTS (mean and percent change among subjects) (Table 1):

TABLE 1

| Hormone | Baseline | Week 4 | % Change |
|---|---|---|---|
| Female DHEA | 162.46 | 763.47 | 370 |
| Female Testosterone | 31.73 | 55.12 | 74 |
| Male DHEA | 179.45 | 463.45 | 158 |
| Male Testosterone | 61.78 | 76.3 | 23 |

CORTISOL Results (mean and percent change among all subjects) (Table 2):

TABLE 2

| Hormone | Baseline | Week 4 | % Change |
|---|---|---|---|
| Female AM Cortisol | 17.19 | 9.17 | −47 |
| Female PM Cortisol | 5.204 | 2.81 | −46 |
| Male AM Cortisol | 13.19 | 14.41 | 9 |
| Male PM Cortisol | 1.54 | 1.78 | 16 |

Quality of Life Scores (rated from 0-10, reported as mean percent change in all subjects studied (Table 3)

TABLE 3

| QOL Parameter | % Change |
|---|---|
| Energy | 159 |
| Satisfaction | 109 |
| Sex Drive | 64 |
| Motivation | 99 |
| Happiness | 102 |

The average increase in recirculating DHEA levels was 370%; testosterone increases an average of 74% and cortisol levels declined by 47% in the female subject group. In the male group the results were not as profound but just as promising. The DHEA levels increased by an average of 158%, testosterone by 23% while cortisol levels increased slightly.

This increase in DHEA levels also led to a corresponding increase in energy levels by 159% as reported by all female subjects, as well as an increase in motivation 99%, satisfaction 109%, happiness 102%, and sex drive by 64% in all of the female subjects who completed the Quality of Life questionnaire.

Increased DHEA may further be employed for treating conditions or diseases related to, or associated with, low testosterone levels, such as loss of muscle mass. Increasing testosterone can have a big impact on energy level, libido and lean muscle mass or body composition especially when an individual's testosterone level is low. Green coffee extract, GCA®, increased testosterone in both men and woman, specifically in those subjects who were on the lower or deficient in recirculating levels. Artificially raising a man's level of testosterone through hormone replacement therapy can increase red blood cell counts and enlarge the prostate, therefore having a way to naturally increase the metabolic pathway toward testosterone optimization can provide a much safer alternative. Artificially raising the level of testosterone in woman can also have some negative side effects such as facial hair growth and mood swings however increasing the female body's natural pathway toward testosterone production can balance hormone and provide a much higher quality of life when deficient. In example 2 subjects taking GCA®, green coffee extract, demonstrated an increase in muscle mass and strength, as well as energy, further supporting the beneficial secondary effects of boosting testosterone naturally.

The beneficial effects subjects experienced when orally ingesting GCA®; green coffee extract on mood, disposition and motivation was further evaluated. A panel of neurotransmitter tests was completed on 8 subjects. Since discovering the beneficial effects GCA® can have on increasing DHEA, essentially by activating or enhancing adrenal function, analysis can be performed on how these hormones help the body respond to enhanced brain function. Cortisol and steroid hormones are produced in the adrenal cortex, while catecholamine's including epinephrine, norepinephrine and dopamine are produced in the adrenal medulla. Stimulating this adrenal function can impact both pathways and assist the body in balancing both steroid hormones and NT function. The impact of balanced NT function is less fatigue, less cravings, anxiousness, and overall better mood. This was supported by the positive changes in all three neurotransmitters, dopamine, epinephrine and norepinephrine with all eight subjects tested before and then 2 weeks after ingestion, thus such increased DHEA may associated with treating conditions such as, or related to, mood disorders and/or neurotransmitter functions.

### Example 2

A 22-week crossover study was conducted to examine the efficacy and safety of a commercial green coffee extract product GCA® at reducing weight and body mass in 16 overweight adults. Subjects received high-dose GCA® (1050 mg), low-dose GCA® (700 mg), or placebo in separate six-week treatment periods followed by two-week washout periods to reduce any influence of preceding treatment. Treatments were counterbalanced between subjects. Primary measurements were body weight, body mass index, and percent body fat.

Significant reductions were observed in body weight (−8.04±2.31 kg), body mass index (−2.92±0.85 kg/m2), and percent body fat (−4.44%±2.00%). A thorough investigation of the data demonstrates that decreases in weight loss occurred when subjects were taking GCA® in both high dose and low dose indications as well as during the washout period. (Table 5). Upon investigation DHEA appears to maintain at an elevated state in subjects for at least the period of the washout (2 weeks) aiding in enhanced energy and metabolic function without the administration of the active (table 6). This provides further evidence to the historical theory that GCA, or its active components, the chlorogenic acids do not positively impact weight loss by the management of the glucose-6-phosphatase pathway but rather through the boost in elevated DHEA levels and/or decreased Cortisol levels. GCA's active components, the chlorogenic acids, are extensively metabolized by humans with a half-life of less than 12 hours. Therefore within a 24-hour period of ingestion, GCA will halt any immediate impact it has on the absorption and or ingestion of carbohydrates. This is further justified by a review of the dose dependent response the chlorogenic acids have on the plasma glucose levels, which is indicative of the positive and direct correlation administration of chlorogenic acid on plasma glucose loading; a higher dose corresponds to a higher change, a lower dose response to a lower change, a placebo dose corresponds to zero change.

### Results:

TABLE 4
Characteristics at baseline and end of study, of 16 overweight subjects.

| | Baseline | End of Study | Difference | |
|---|---|---|---|---|
| | (Week 0) | (Week 22) | (Week 22 − Week 0) | |
| Characteristic | M ± SD | M ± SD | M ± SD | Change |
| Weight (kg) | 76.69 ± 7.91 | 68.65 ± 7.78 | 8.04 ± 2.31** | −10.5% |
| BMI (kg/m²) | 28.22 ± 0.91 | 25.25 ± 1.19 | 2.92 ± 0.85** | −10.3% |
| BIA | 28.13 ± 4.95 | 23.69 ± 4.95 | 4.44 ± 2.00** | −15.8% |
| DHEA | 142.76 ± 78.15 | 547.02 ± 303.42 | 424.26 ± 190.7* | +323% |

Note.
BMI = Body Mass Index; BIA = Bioelectrical Impedance Analysis; DHEA = Dehydroepiandrosterone.
**p < .0001,
*p < .005

TABLE 5
Characteristics at the start and the end of each treatment arm for 16 overweight subjects.

| | HD Arm | | LD Arm | | PL Arm | |
|---|---|---|---|---|---|---|
| | Start | End | Start | End | Start | End |
| | M ± SD | M ± SD | M ± SD | M ± SD | M ± SD | M ± SD |
| Characteristic | (95% CI) | (95% CI) | (95% CI) | (95% CI) | (95% CI) | (95% CI) |
| Weight (kg) | 72.86 ± 8.91 | 70.82 ± 8.40 | 71.25 ± 7.30 | 69.71 ± 7.30 | 72.15 ± 8.64 | 72.47 ± 8.47 |
| | (68.11-77.61) | (66.34-75.30) | (67.36-75.14) | (65.82-73.60) | (67.55-76.75) | (67.96-76.98) |
| BMI (kg/m²) | 26.78 ± 1.55 | 26.03 ± 1.36 | 26.25 ± 1.37 | 25.66 ± 1.20 | 26.55 ± 1.96 | 26.67 ± 1.72 |
| | (25.95-27.61) | (25.31-26.75) | (25.52-26.98) | (25.02-26.30) | (25.51-27.59) | (25.75-27.59) |
| BIA (% body | 25.94 ± 5.35 | 24.75 ± 5.20 | 25.94 ± 4.99 | 24.88 ± 4.99 | 25.88 ± 5.40 | 25.00 ± 5.52 |
| fat) | (23.09-28.79) | (21.98-27.52) | (23.28-28.60) | (22.22-27.54) | (23.00-28.76) | (22.20-27.82) |

TABLE 6
DHEA Levels at the start and the end of each treatment arm for 16 overweight subjects.

| | | HD Arm | | LD Arm | | PL Arm | |
|---|---|---|---|---|---|---|---|
| | Baseline | Start | End | Start | End | Start | End |
| | M ± SD | M ± SD | M ± SD | M ± SD | M ± SD | M ± SD | M ± SD |
| Characteristic | (95% CI) | (95% CI) | (95% CI) | (95% CI) | (95% CI) | (95% CI) | (95% CI) |
| DHEA | 142.76 ± 78.15 | 272.77 ± 108.91 | 670.82 ± 218.40 | 371.25 ± 127.30 | 569.71 ± 197.56 | 302.15 ± 98.64 | 352.47 ± 108.47 |

Note.
HD = High Dose Green Coffee Extract;
LD = Low Dose Green Coffee Extract;
PL = Placebo; BMI = Body Mass Index;
BIA = Bioelectrical Impedance Analysis;
DHEA = Dehydroepiandrosterone.

### Example 3

The same 22-week crossover study that was conducted to examine the efficacy and safety of a commercial green coffee extract product GCA® at reducing weight and body mass in 16 overweight adults also monitored 5 male patients who exhibited male pattern baldness for changes in scalp hair loss during the entire treatment period. Subjects were monitored during the high-dose GCA® (1050 mg), the low-dose GCA® (700 mg), and the placebo in separate six-week treatment periods followed by two-week washout periods to reduce any influence of preceding treatment. Treatments were counterbalanced between subjects.

Scalp examination was performed in two stages; first an inspection of the scalp for inflammation, scaling, erythema, and follicular opening was preformed at the onset of the study. Measurement of the initial scalp density, hair thickness and appearance were noted. At the end of the 22 weeks an examination of the distribution, pattern and density of the hair was completed and the quality of the hair shaft in terms of density, length and overall appearance was completed.

TABLE 7
Baseline

| | | | | Baseline | |
|---|---|---|---|---|---|
| | | Density | Hair | Hamilton- | |
| Sub- | Inflam- | Score | Thickness | Norwood Scale | Overall |
| ject | mation | (fu/cm2) | (um) | (Norwood 1975) | Appearance |
| 1 | Not | 0 | na | NA | bald no |
| | present | | | | hair |
| | | | | | regrowth |
| 2 | not | 0 | na | NA | bald no |
| | present | | | | active |
| | | | | | regrowth |
| 3 | not | 22 | 30 | VII | 90% bald |
| | present | | | | shunted |
| | | | | | hair growth |
| 4 | not | 66 | 34 | VA | 50% bald |
| | present | | | | active |
| | | | | | hair growth |
| 5 | not | 56 | 32 | VI | 82% bald |
| | present | | | | shunted |
| | | | | | hair growth |

TABLE 8
At 22 weeks

| | | Density | Hair | Linear Hair | |
|---|---|---|---|---|---|
| Sub- | Inflam- | Score | Thickness | Growth rate | Overall |
| ject | mation | (fu/cm2) | (um) | (cm) | Appearance |
| 1 | not | 12 | 30 | 2.2 | 10% active |
| | present | | | | hair growth |
| 2 | not | 6 | na | 0.65 | slight active |
| | present | | | | regrowth |
| 3 | not | 31 | 32 | 3.2 | 87% active |
| | present | | | | hair growth |
| 4 | not | 82 | 36 | 3.7 | 36% active |
| | present | | | | hair growth |
| 5 | not | 67 | 36 | 3.4 | 70% active |
| | present | | | | hair growth |

The results indicate that all five subjects who experienced some type of male pattern baldness had improvements in both the thickness of the hair as well as the growth of the hair throughout the duration of the 22-week study. The average density improved 28% and the average thickness by 8% indicating and increase in hair health as well physical appearance. The length increased an average of 2.63 cm in 22 weeks. It is presumed that the increase in hair growth is a direct function of the elevated levels of recirculating DHEA levels seen in the subjects.

### Example 4

Forty two female subjects with a Fitzpatrick skin type between I-III were randomly assigned into four different participant groups. The four groups were requested to apply one of the following topical regimen protocols daily for 12 weeks:

1. Group 1 (Brown)—control group applying a Neutrogena Moisturizer 2× daily and Tretinoin Cream 0.025% applied 1× daily.
2. Group 2 (Green)—group applying a Green coffee extract, CGA® 2× daily.

Both groups were also asked to cleanse their face 2× daily prior to application of the serums, in addition the am routine included applying sunscreen spf 30 or greater. All subjects visited the Raval Aesthetician clinic every four weeks. Throughout the course of the clinical and upon each visit each subject was given a skin analysis with Visia® technology, requested to complete a performance questionnaire and photographs were taken to monitor skin progress in visual appearance.

The Visia® analysis reported the following parameters: change in facial spots, UV damage, wrinkles, texture, pores and porphiryns. Data provided a snapshot of the individuals skin performance relative to other subjects within the database of the same skin type as well as scoring assessment for each parameter to measure against that subjects baseline at time t=0.

The performance questionnaire contained questions each subject experience rating the following parameters on a scale 1-5: overall appearance, skin tone, skin clarity, skin texture, moisture and skin elasticity.

All data with the exception of the level of porphiryns was analyzed relative to the control and corrected for ANNOVA to standard for an individual's baseline as well as significant changes with group outcome. Porphiryns were excluded in most analysis relative to control due to issues with product handling and lack of preservatives within the product which promoted elevated bacterial counts that would presumably not be present had topical contained additional ingredients to control bacteria and product contamination upon handling.

Visia® analysis (Table 9) demonstrated an improvement of at least three skin parameters measured for all subjects: spots, UV damage and pores. Addressing each parameter an improvement in skin spots was most prominent within the green coffee extract (GCA®) group by 8%. UV damage which was a critical measurement in performance outcome demonstrated the most promising benefits in both the green coffee extract, GCA® (5%).

TABLE 9
Visia ® Analysis. Data reported on average percent change
relative to individual's baseline, (p < .005)

| | Control | GCA |
|---|---|---|
| Spot Reduction | 4% | 6% |
| UV Damage Reduction | 1% | 5% |
| Wrinkle Reduction | −10% | 22% |
| Texture Improvement | −1% | 12% |
| Pore Reduction | 7% | 10% |

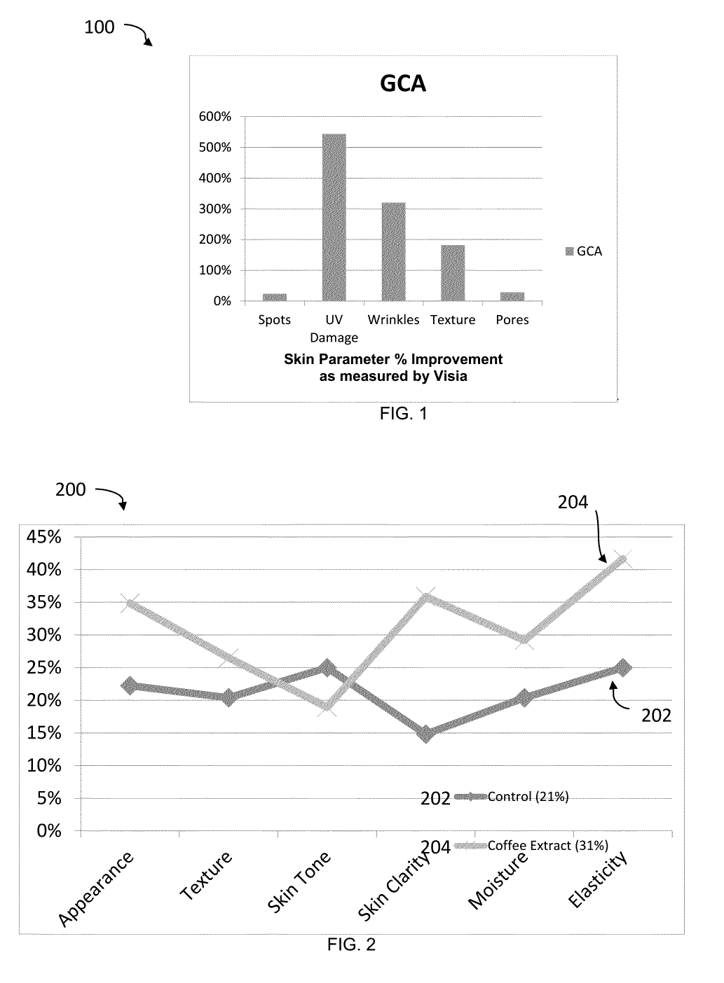

Relative to the control, the GCA® topical had statistically significant benefits in skin performance outcome (FIG. 1) for UV damage, wrinkles and skin texture. There was a strong and positive correlation between the change in UV damage and the amount of wrinkles and overall skin texture as measured by Visia® leading to the link between antioxidant benefit/performance and long term skin health and visual repair.

FIG. 1 is a chart 100 of a Visia® analysis relative to control group for each skin parameter measured. Data reported on average percent increase in skin parameter measured relative to the control group corrected for individuals baseline values (p<0.005).

Important to long term compliance and ultimately product performance is the perception of skin improvements with the subjects. Therefore a skin performance questionnaire become an integrate part of the review association with skin performance and perception of value to the subject. As was seen within the Visia® analysis the survey results also are indicative of a statistically significant improvement in all skin parameters assessed by each individual. The group using the Green Coffee Extract objectively scored an overall 31% improvement relative to their baseline reporting, with significant improvements in skin clarity and elasticity.

TABLE 10
Table 10: Product Quality Questionnaire as assessed by the
individual. Data reported on average percent increase in performance
of skin parameter, (p < .002) against individuals baseline data value.

| | Control (21%) | Coffee Extract (31%) |
|---|---|---|
| Appearance | 22% | 35% |
| Texture | 20% | 27% |
| Skin Tone | 25% | 19% |
| Skin Clarity | 15% | 36% |
| Moisture | 20% | 29% |
| Elasticity | 25% | 42% |

Chart 200 of FIG. 2 depicts statistics resulting from a produce quality questionnaire as assessed by the individual for a control group 202 and a coffee extract group 204. Chart 200 indicates a visual perspective of product performance for each skin parameter for each product applied. Overall, all subjects also reported an increase in product performance relative to both their baseline data and the control group, with the order of improvement of each topical being Control, Glucarate, Combination, Green Coffee Extract groups.

Data reported on average percent increase in performance of skin parameter, (p<0.002) against individuals baseline data value. Relative to the control group there were varying changes in the product performance described by the individual.

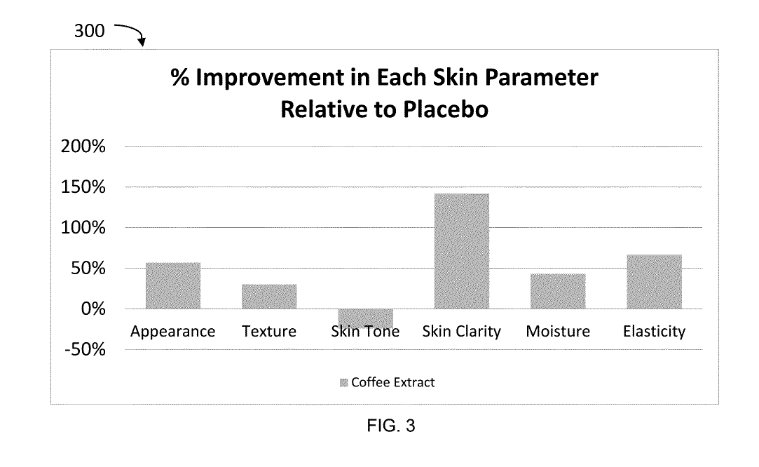

Chart 300 of FIG. 3 is a subject questionnaire results reporting product performance for each skin parameter. Data is expressed as average value relative to control for each skin parameter (p<0.002).

It has been reported within the literature that topical application of a low dose retinoic acid applied daily can have positive benefits to skin performance through enhanced skin cell turnover and ultimately lead to skin repair. Specifically, as it relates to the negative effects of photo damage retinoic acid has been shown when applied topically, to decrease UV damage, increase skin collagen, and reduce wrinkles. We have been able to demonstrate that applying additional topical antioxidants in the form of green coffee beans, GCA® can have an enhanced effect on this current dermatology treatment. In addition to reducing the effects of photo damage, the green coffee extract showed significant improvements in skin clarity and added moisture, resulting in enhance skin elasticity.

In conclusion, green coffee bean extract, GCA® has been shown to positively impact skin performance, skin aging and the visual impact on skin health. Specifically, clinical review demonstrated that topical application of antioxidants in the form of green coffee extracts standardized to 50% chlorogenic acids provided a 32% increase in healthy skin parameters as measured by Visia® analysis. The most significant benefit came with applying the green coffee extract resulting in reduction in UV damage (543%) and wrinkles (320%) in comparison with a topical containing Tretinoin Cream 0.05%. GCA®, green coffee extract at 6 w/w % as a topical agent, was well tolerated. No adverse events and skin conditions were reported.

Although the invention has been described with reference to specific embodiments, this description is not meant to be construed in a limited sense. Various modifications of the disclosed embodiments, as well as alternative embodiments of the invention will become apparent to persons skilled in the art upon the reference to the description of the invention. It is, therefore, contemplated that the appended claims will cover modifications that fall within the scope of the invention.

What is claimed is:

1. A method for elevating naturally recirculating dehydroepiandrosterone (DHEA) levels in a human subject, said method comprising:
generating a green coffee extract containing at least 20% chlorogenic acids by dry weight;
generating a composition which includes said green coffee extract and is administrable to a human subject; and
administering a therapeutically effective amount of said composition to said human subject, thereby elevating naturally recirculating DHEA levels.

2. The method of claim 1 wherein a dosage of the green coffee extract is provided in a range from approximately 1 mg to approximately 4000 mg per dosage.

3. The method of claim 1 wherein an optimum dosage unit is provided in a range from approximately 250 mg to approximately 400 mg per dosage.

4. The method of claim 1 wherein the composition is administered orally.

5. The method of claim 1 wherein the composition is administered topically.

6. The method of claim 1 wherein the chlorogenic acids and a related compounds of said green coffee extract include:
3-CQA=2%-15%
5-CQA=5%-25%
4-CQA=2%-15%
5-FQA=1%-12%
3,4-diCQA=1%-12%
3,5-diCQA=1%-12%
4,5-diCQA=1%-12%.

7. The method of claim 1, further comprising treating a condition of said human subject, said condition being selected from the group comprising a low energy level, a low libido, an increased body mass index, an increased percent body fat, a disease or condition associated with adrenal fatigue and/or insulin resistance.

8. The method of claim 7, wherein a dosage of the green coffee extract is provided in a range from approximately 1 mg to approximately 4000 mg per dosage.

9. The method of claim 7, wherein an optimum dosage unit is provided in a range from approximately 250 mg to approximately 400 mg per dosage.

10. The method of claim 7, wherein the composition is administered orally.

11. The method of claim 1 wherein said elevated DHEA level enhances hair growth.

12. The method of claim 1 wherein said elevation of DHEA level ameliorates symptoms related to decreased energy level, decreased libido, decreased motivation and decreased overall happiness and life satisfaction.

13. The method of claim 1 wherein said elevation of DHEA level decreases a body mass index and/or percent body fat among said human subjects.

14. A composition for elevating naturally recirculating DHEA level in a human subject, said composition comprising a therapeutically effective amount of green coffee bean extract, said green coffee extract containing at least 20% chlorogenic acids by dry weight.

15. A method to stimulate the production of DHEA in a human subject thereby elevating the naturally recirculating DHEA levels, said method comprises: administering to said subject an effective dose of green coffee bean extract, said extract containing at least 20% chlorogenic acids by dry weight.

16. The method of claim 1, further comprising elevating naturally recirculating testosterone levels of said human subject as a result of administration of said therapeutically effective amount of said composition.

17. The method of claim 1, further comprising treating a condition of said human subject, said condition being a loss of muscle mass or other negative symptoms associated with low recirculating testosterone levels.

18. The method of claim 1, further comprising reducing naturally recirculating cortisol levels of said human subject as a result of administration of said therapeutically effective amount of said composition.

19. The method of claim 1, further comprising treating a condition of said human subject, said condition being a disease or condition associated with at least one of a mood disorder or a neurotransmitter function.
